(19) United States
(12) Patent Application Publication
(10) Pub. No.: US 2026/0183216 A1
(43) Pub. Date: Jul. 2, 2026

(54) GRANULE COMPOSITION WITH EASY RINSING AND SOOTHING OIL CONTROL PROPERTIES AND APPLICATION THEREOF

(71) Applicant: Guangzhou Beaver Cosmetics Co., Ltd., Guangzhou (CN)

(72) Inventor: Hongpeng Zhang, Guangzhou (CN)

(21) Appl. No.: 19/223,013

(22) Filed: May 29, 2025

(30) Foreign Application Priority Data
Dec. 30, 2024 (CN) 202411965581.2

(51) Int. Cl. A61K 8/73; A61K 8/02; A61K 8/41; A61K 8/46; A61Q 5/02; A61Q 19/10

(52) U.S. Cl. CPC A61K 8/737; A61K 8/0241; A61K 8/416; A61K 8/466; A61Q 5/02; A61Q 19/10

(57) ABSTRACT

This disclosure belongs to the technical field of daily chemical products, and in particular relates to a granule composition with easy rinsing and soothing oil control properties and applications thereof. The granule composition includes the following components in parts by weight: 21-73 parts of a surfactant blend, 2.1-21 parts of a humectant, 1-4.9 parts of a soothing ingredient, 10-20 parts of a filler, 1-5 parts of an anti-caking agent and 0.1-1 part of a thickener; where the surfactant blend is a formulated blend of distearyl dimethyl ammonium chloride, sodium C14-16 olefin sulfonate, sodium cocoyl hydroxyethylsulfonate and sodium lauroyl glutamate. In this disclosure, the specified surfactant blend is combined with other components to provide a solid granule which has advantages of multiple soothing and cleansing effects, reduced need for preservatives, long-term stability and excellent use effects, easy rinsing performance without residue, easy transportation or carrying.

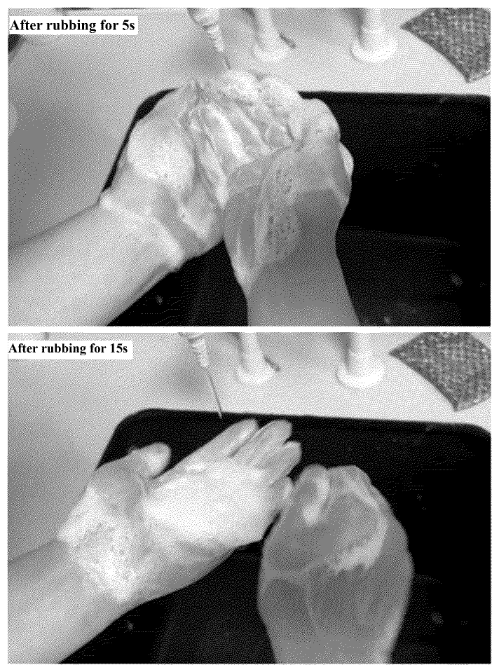

### CROSS-REFERENCE TO RELATED APPLICATION

This disclosure claims the benefit of Chinese patent application No. 202411965581.2, filed on Dec. 30, 2024, which is incorporated herein by reference in its entirety.

### TECHNICAL FIELD

This disclosure belongs to the technical field of daily chemical products, and in particular relates to a granule composition with easy rinsing and soothing oil control properties and applications thereof.

### BACKGROUND

At present, traditional liquid cleansing and caring products are widely used in the market, but they have many problems in their usage process. Firstly, many liquid cleansing and caring products contain high levels of chemical components that can disrupt the natural barriers of the skin and hair, leading to dryness and tightness, especially for consumers with dry or sensitive skin. Secondly, preservatives, alcohol or other additives in some cleansing and caring products may cause allergic reactions to the skin, resulting in uncomfortable symptoms such as redness, swelling and itching, especially sensitive people are more susceptible.

In addition, the production process of liquid cleansing and caring products also faces environmental protection and resource consumption issues. The production of liquid cleansing and caring products requires a large amount of water, and due to complex formulations and multiple packaging of the liquid cleansing and caring products, the waste of water resource is quite severe. Water consumption in the production process, the weight of the liquid cleansing and caring products during the transportation and the waste of water during use will put pressure on water resources, and also increase the carbon footprint and environmental burden.

Accordingly, there is an urgent need to develop a solid cleansing and caring product which has a mild cleansing ability and is easy to rinse without residue. It not only can reduce skin irritation and allergic reactions, but also reduce water consumption. Further, it has environmental advantages and is easy to transport or carry. Therefore, it is in line with the modern consumer's dual needs for both environmental protection and health, and can fill the market gaps.

### SUMMARY

An objective of this disclosure is to overcome the shortcomings of the prior art and provide a granule composition with easy rinsing and soothing oil control properties and applications thereof.

To achieve the above objective, the following technical solutions are adopted.

In a first aspect, provided herein is a granule composition with easy rinsing and soothing oil control properties, wherein the granule composition comprises the following components in parts by weight: 21-73 parts of a surfactant blend, 2.1-21 parts of a humectant, 1-4.9 parts of a soothing ingredient, 10-20 parts of a filler, 1-5 parts of an anti-caking agent and 0.1-1 part of a thickener; the surfactant blend comprises a cationic surfactant, an anionic surfactant and an amino acid surfactant; the cationic surfactant comprises at least one of distearyl dimethyl ammonium chloride and guar gum hydroxypropyl trimethyl ammonium chloride; the anionic surfactant comprises sodium cocoyl hydroxyethionate and sodium C14-16 olefin sulfonate; and the amino acid surfactant comprises sodium lauryl glutamate.

When the specified cationic surfactant, anionic surfactant and amino acid surfactant are combined in this disclosure to prepare a solid granule composition, the various surfactants can cooperate with one another to effectively remove the oil or dirt on the surface of the skin, improve the oil control and cleansing efficacy of the composition, and maintain the water and oil balance of the skin or hair, while playing a certain role in moisturizing and moistening so as to reduce dry and tight feelings of the skin after the granule composition is applied to the skin. In addition, the interaction among the specified surfactants can enable the solid granule composition to generate a richer and finer foam during use, which is quickly dissolved and easily rinsed, effectively improving the rinsing efficiency of the solid granule composition.

Through experimental research, it has been found that the solid granule composition prepared by reasonably combining the surfactant blend with a humectant and a soothing ingredient can achieve a full balance in terms of oil control and cleansing efficacy, moisturizing, soothing, rich foam and easy rinsing property etc. Compared with the traditional liquid cleansing and caring products, the solid granule composition of the disclosure can maintain a long-term stability of a product including the same without any preservatives, avoid skin itching and allergic reactions caused by preservatives and other additives, and is suitable for customers with sensitive skin. Further, the granule composition can significantly reduce water consumption, and has a decreased volume so that it is easy to transport or carry. Meanwhile, the solid granules with a relatively low water activity can effectively maintain the stability of the active soothing ingredients therein, thus providing a more ideal usage experience for consumers.

Preferably, the granule composition comprises the following components in parts by weight: 30.5-57.5 parts of the surfactant blend, 8.3-18.8 parts of the humectant, 1.9-3.7 parts of the soothing ingredient, 15-20 parts of the filler, 2-4 parts of the anti-caking agent and 0.2-0.8 part of the thickener.

More preferably, the granule composition comprises the following components in parts by weight: 50.5 parts of the surfactant blend, 14.5 parts of the humectant, 2.6 parts of the soothing ingredient, 20 parts of the filler, 3 parts of the anti-caking agent and 0.5 part of the thickener.

Through experimental research, it has been found that when the components of the disclosure are combined in the above optimal ratio, the use effect of the prepared granule composition is the best. Besides, the water activity of the granule composition is relatively lower, so that the stability and solubility of the composition can be significantly improved, and the use effect of the product can be enhanced to the greatest extent.

In an embodiment, the surfactant blend is a formulated blend of distearyl dimethyl ammonium chloride, sodium C14-16 olefin sulfonate, sodium cocoyl hydroxyethylsulfonate and sodium lauroyl glutamate; and a mass ratio of the distearyl dimethyl ammonium chloride/sodium C14-16 olefin sulfonate/sodium cocoyl hydroxyethionate/sodium lauroyl glutamate is (0.1-3):(1-10):(15-35):(5-25).

Preferably, the mass ratio of the distearyl dimethyl ammonium chloride/sodium C14-16 olefin sulfonate/sodium cocoyl hydroxyethionate/sodium lauroyl glutamate is (0.5-2.5):(2-5):(20-35):(8-15).

More preferably, the mass ratio of the distearyl dimethyl ammonium chloride/sodium C14-16 olefin sulfonate/sodium cocoyl hydroxyethionate/sodium lauroyl glutamate is 2:3.5:35:10.

Through experimental research, it has been found that when the above four surfactants are combined in a specified ratio in this disclosure, the cleansing and dirt-removing efficacy of the granule composition can be significantly enhanced, so that the granule composition possesses mild cleansing and oil control properties. Besides, the granule composition possesses enhanced hydration, and plays a certain role in moisturizing and moistening, reducing dryness and tightness of the skin or hair after deep cleansing.

In an embodiment, the soothing ingredient comprises allantoin, dipotassium glycyrrhizinate, a chamomile (Matricaria chamomilla L.) extract and a calendula (Calendula officinalis L.) extract; and a mass ratio of allantoin/dipotassium glycyrrhizinate/the chamomile extract/the calendula extract is (0.5-2):(0.3-0.9):(0.1-1):(0.1-1).

Preferably, the mass ratio of allantoin/dipotassium glycyrrhizinate/the chamomile extract/the calendula extract is (0.8-1.5):(0.5-0.8):(0.3-0.7):(0.3-0.7).

Moor preferably, the mass ratio of allantoin/dipotassium glycyrrhizinate/the chamomile extract/the calendula extract is 1:0.6:0.5:0.5.

Through experimental research, it has been found that when the chamomile extract and the calendula extract are reasonably combined with allantoin and dipotassium glycyrrhizinate, as the soothing ingredient, to prepare the granule composition, the soothing effect of the composition can be significantly enhanced, avoiding post-cleansing discomfort such as dryness and tightness. Notably, dipotassium glycyrrhizinate, the chamomile extract and the calendula extract exhibit a pronounced synergistic effect which effectively relieves irritation caused by deep cleansing agents while providing additional soothing, moisturizing and protective benefits, thereby providing a milder cleansing experience. When any one or more of the three ingredients are absent, the prepared granule composition has significantly reduced soothing and moisturizing effect, resulting in a dry and tight feeling after cleansing.

In an embodiment, the humectant comprises sodium hyaluronate, trehalose and sorbitol; and/or, the filler comprises modified corn starch; and/or, the anti-caking agent comprises magnesium stearate; and/or, the thickener comprises hydroxypropyl methyl cellulose.

In an embodiment, a mass ratio of sodium hyaluronate/trehalose/sorbitol is (0.1-1):(1-10):(1-10).

Preferably, the mass ratio of sodium hyaluronate/trehalose/sorbitol is (0.3-0.8):(5-10):(3-8).

More preferably, the mass ratio of sodium hyaluronate/trehalose/sorbitol is 0.5:9:5.

In an embodiment, the granule composition further comprises the following components in parts by weight: 0.2-0.8 part of a pH regulator and 0.5-1.5 parts of a fragrance.

Preferably, the pH regulator comprises citric acid; and/or, the fragrance comprises an essence.

In a second aspect, provided herein is a preparation method of the granule composition, comprising the following steps:

mixing all the components uniformly to give a mixture, pulverizing the mixture into powder, compressing the powder under a pressure of 50-80 MPa at room temperature to form compressed granules, drying the compressed granules at 50-60° C. for 4-6 hours, and cooling the compressed granules to give the granule composition.

The solid granule composition herein is prepared through processes of conventional mixing, compression molding, and drying. These processes do not adversely affect the finished product's soothing and oil control efficacy while significantly enhancing transportation and usage convenience. Compared with traditional liquid products, the solid granule composition has advantages such as compact size, safer transportation, high efficiency, portability, and environmental friendliness.

In a third aspect, provided herein is use of the granule composition in the preparation of cleansing and caring products.

In a fourth aspect, provided herein is a cleansing and caring product, comprising the granule composition.

Preferably, the cleansing and caring product is selected from a shampoo product, a facial cleansing product, and a bath product.

The granule composition with easy rinsing and soothing oil control properties of this disclosure is suitable for personal care products such as a shampoo product, a facial cleansing product and a bath product. It can efficiently remove dirt and oil from skin surfaces due to superior solubility and oil control efficacy thereof, and it will not cause irritation to skin or hair due to soothing and moisturizing properties thereof, thus it is suitable for all types of skins and hairs. The solid form facilitates storage and transportation, reduces the need for preservatives which are usually applied in liquid products, avoids the irritation by chemical ingredients such as preservatives, and provides a more environmentally friendly, safer and more efficient usage experience.

In a fifth aspect, provided herein is a shampoo granule with easy rinsing, soothing and smoothing properties, comprising the granule composition of this disclosure, a softener and a hair regulator.

Preferably, the softener comprises at least one of acanthus kernel oil, Jojoba seed oil and coconut oil; and the hair regulator comprises lactobacillus/rye flour ferment filtrate.

In the disclosure, the shampoo granule can be prepared just by adding hair care factors with specific functions to the granule composition to give a mixture, and subjecting the mixture to the above process. The preparation method is simple, and the active ingredients such as hair care factors can be effectively wrapped in the shampoo granule, which can avoid degradation or inefficiency of the active ingredients during the preparation process or long-term storage of the shampoo granule, thereby improving the stability and overall performance of the shampoo granule.

Compared with the prior art, this disclosure has the following beneficial effects:

In this disclosure, a solid granule composition is innovatively developed by combining the selected specific surfactant blend with other components, using the above preparation method. The components of the granule composition cooperate with one another to play multiple roles in soothing, cleansing and oil control, providing excellent mild and nourishing effects. In addition, compared with the traditional liquid cleansing and caring products, the solid granule composition effectively reduces the need for preservatives, ensures the long-term stability and use effect of the product comprising the same. It is easy to rinse without residues, significantly reduces water consumption, reduces the consumption of water resources, greatly reduces the bulk thereof, and is easy to transport or carry. It is widely used in cleansing and caring products such as a shampoo product, a facial cleansing product or a bath product.

### BRIEF DESCRIPTION OF THE DRAWINGS

The sole FIGURE shows the foaming performance of the shampoo granule obtained in Application Example 1 under different rubbing durations.

### DETAILED DESCRIPTION

To better explain the objectives, technical solutions and advantages of the disclosure, specific examples are provided to further explain the disclosure. It should be understood by those skilled in the art that the specific examples are provided only to explain the disclosure, and are not intended to limit it.

Raw materials involved in the following examples and comparative examples are commercially available materials, unless otherwise specified. The experimental operations not specifically stated herein are routine operations in this field, or can be understood or known by those skilled in the art based on prior art or common knowledge.

### Examples 1-7

Granule compositions with easy rinsing and soothing oil control properties were provided in Examples 1-7, specific formulations thereof were shown in Table 1. The preparation method of the granule compositions comprised the following steps:

all the components were mixed evenly and then ground into powder, the powder was compressed under 65 MPa pressure at room temperature to form compressed granules, and then the compressed granules were dried at 55° C. for 5 h, then cooled to give the granule compositions.

TABLE 1
Formulations (in parts by weight) of the granule compositions in Examples 1-7

| | Component | Ex. 1 | Ex. 2 | Ex. 3 | Ex. 4 | Ex. 5 | Ex. 6 | Ex. 7 |
|---|---|---|---|---|---|---|---|---|
| Surfactant | Distearyl dimethyl | 2 | 0.1 | 3 | 0.5 | 2.5 | 5 | 2 |
| blend | ammonium chloride | | | | | | | |
| | Sodium C14-16 | 3.5 | 1 | 10 | 2 | 5 | 15 | 3.5 |
| | olefins sulfonate | | | | | | | |
| | Sodium cocoyl | 35 | 15 | 35 | 20 | 35 | 5 | 35 |
| | hydroxyethylsulfonate | | | | | | | |
| | Sodium lauroyl | 10 | 5 | 25 | 8 | 15 | 25.5 | 10 |
| | glutamate | | | | | | | |
| Humectant | Sodium hyaluronate | 0.5 | 0.1 | 1 | 0.3 | 0.8 | 0.5 | 0.5 |
| | Trehalose | 9 | 1 | 10 | 5 | 10 | 9 | 9 |
| | Sorbitol | 5 | 1 | 10 | 3 | 8 | 5 | 5 |
| Soothing | Allantoin | 1 | 0.5 | 2 | 0.8 | 1.5 | 1 | 0.1 |
| ingredient | Dipotassium | 0.6 | 0.3 | 0.9 | 0.5 | 0.8 | 0.6 | 1.2 |
| | glycyrrhizinate | | | | | | | |
| | Chamomile extract | 0.5 | 0.1 | 1 | 0.3 | 0.7 | 0.5 | 1.2 |
| | Calendula extract | 0.5 | 0.1 | 1 | 0.3 | 0.7 | 0.5 | 0.1 |
| Filler | Modified corn starch | 20 | 10 | 20 | 15 | 20 | 20 | 20 |
| Anti-caking | Magnesium stearate | 3 | 1 | 5 | 2 | 4 | 3 | 3 |
| agent | | | | | | | | |
| pH | Citric acid | 0.5 | 0.2 | 0.8 | 0.3 | 0.6 | 0.5 | 0.5 |
| regulator | | | | | | | | |
| Fragrance | (daily) essence | 1 | 0.7 | 1.3 | 1 | 1 | 1 | 1 |
| Thickener | Hydroxypropyl | 0.5 | 0.1 | 1 | 0.2 | 0.8 | 0.5 | 0.5 |
| | methyl cellulose | | | | | | | |

Note:
Ex. represents “Example”.

### Example 8

A solid granule composition with easy rinsing and soothing oil control properties was provided in this example, which differed from Example 1 in the preparation method. The preparation method in this example comprised the following steps:
S1. the components except the thickener were weighed according to the formulation of Example 1, and then mixed evenly, dried, and ground into powder;
S2. the thickener was weighed according to the formulation of Example 1, then mixed with water to give a thickener aqueous solution with a mass concentration of 0.5%;
S3. the powder obtained in step S1 was added to a granulator, and then the thickener aqueous solution obtained in step S2 was sprayed into the granulator, and a temperature of the air blown by a blower into the granulator was controlled at 85° C. with a speed of the blower of 15 Hz, so that the powder was turned up and down in the granulator to fully contact with the sprayed aqueous solution, thereby forming granules; wherein the outlet air temperature of the granulator was controlled at 30° C. and the humidity in the granulator was controlled at 30%; preferably, the thickener aqueous solution was evenly sprayed into the granulator by using a peristaltic pump;
S4. target granules within a 10-100 mesh range were sieved as the granule composition for filling to give the finished product.

### Comparative Examples 1-9

Formulations of the granule compositions in Comparative Examples 1-9 were provided, as shown in Table 2. The preparation methods of the granule compositions provided in Comparative Examples 1-9 were the same as that of Examples 1-5, except the formulations.

TABLE 2
Formulations (in parts by weight) of the granule compositions in Comparative Examples 1-9

| | Component | CEx. 1 | CEx. 2 | CEx. 3 | CEx. 4 | CEx. 5 | CEx. 6 | CEx. 7 | CEx. 8 | CEx. 9 |
|---|---|---|---|---|---|---|---|---|---|---|
| Surfactant | Distearyl dimethyl | / | 2 | 5.5 | / | 5.5 | 2 | 2 | 2 | 2 |
| blend | ammonium chloride | | | | | | | | | |
| | Sodium C14-16 | 5.5 | / | 10 | 20.5 | / | 3.5 | 3.5 | 3.5 | 3.5 |
| | olefins sulfonate | | | | | | | | | |
| | Sodium cocoyl | 35 | 35 | 35 | / | 45 | 35 | 35 | 35 | 35 |
| | hydroxyethylsulfonate | | | | | | | | | |
| | Sodium lauroyl | 10 | 13.5 | / | 30 | / | 10 | 10 | 10 | 10 |
| | glutamate | | | | | | | | | |
| Humectant | Sodium hyaluronate | 0.5 | 0.5 | 0.5 | 0.5 | 0.5 | 0.5 | 0.5 | 0.5 | 0.5 |
| | Trehalose | 9 | 9 | 9 | 9 | 9 | 9 | 9 | 9 | 9 |
| | Sorbitol | 5 | 5 | 5 | 5 | 5 | 5 | 5 | 5 | 5 |
| Soothing | Allantoin | 1 | 1 | 1 | 1 | 1 | 1 | 1 | 1 | 1 |
| ingredient | Dipotassium | 0.6 | 0.6 | 0.6 | 0.6 | 0.6 | 0.9 | 0.9 | / | 1.6 |
| | glycyrrhizinate | | | | | | | | | |
| | Chamomile extract | 0.5 | 0.5 | 0.5 | 0.5 | 0.5 | / | 0.7 | 0.8 | / |
| | Calendula extract | 0.5 | 0.5 | 0.5 | 0.5 | 0.5 | 0.7 | / | 0.8 | / |
| Filler | Modified corn starch | 20 | 20 | 20 | 20 | 20 | 20 | 20 | 20 | 20 |
| Anti-caking | Magnesium stearate | 3 | 3 | 3 | 3 | 3 | 3 | 3 | 3 | 3 |
| agent | | | | | | | | | | |
| pH | Citric acid | 0.5 | 0.5 | 0.5 | 0.5 | 0.5 | 0.5 | 0.5 | 0.5 | 0.5 |
| regulator | | | | | | | | | | |
| Fragrance | (daily) essence | 1 | 1 | 1 | 1 | 1 | 1 | 1 | 1 | 1 |
| Thickener | Hydroxypropyl | 0.5 | 0.5 | 0.5 | 0.5 | 0.5 | 0.5 | 0.5 | 0.5 | 0.5 |
| | methyl cellulose | | | | | | | | | |

Note:
CEx. represents “Comparative Example”.

### Comparative Example 10

A solid granule composition was provided in this comparative example, which differed from Example 1 only in that distearyl dimethyl ammonium chloride was replaced with octadecyl dimethyl benzyl quaternary ammonium chloride, which was also a cationic surfactant. The remaining components and dosages thereof were the same as those in Example 1.

### Comparative Example 11

A solid granule composition was provided in this comparative example, which differed from Example 1 only in that the chamomile extract was replaced with aloe vera (Aloe vera (L.) Burm. f.) extract, which was also a soothing ingredient. The remaining components and dosages thereof were the same as those in Example 1.

### Application Examples 1-7

Solid shampoo granules were provided in these application examples, each of which comprised the following components in mass percentages: 99.6% of the granule composition, 0.1% of acanthus kernel oil (ARGANIA SPINOSA), 0.1% of Jojoba seed oil (SIMMONDSIA CHINENSIS), 0.1% of coconut oil (COCOS NUCIFERA) and 0.1% of lactobacillus/rye flour ferment filtrate. The granule compositions used in Application Examples 1-7 respectively corresponded to the granule compositions produced in Examples 1-7.

The preparation method of the shampoo granules comprised the following steps:
all the components were mixed evenly and then ground into powder, the powder was compressed under a pressure of 50-80 MPa at room temperature to form compressed granules, and then the compressed granules were dried at 50-60° C. for 4-6 h, then cooled to give the shampoo granules.

### Application Example 8

A solid shampoo granule was provided in this application example, which comprised the following components in mass percentages: 99.6% of the granule composition, 0.1% of acanthus kernel oil (ARGANIA SPINOSA), 0.1% of Jojoba seed oil (SIMMONDSIA CHINENSIS), 0.1% of coconut oil (COCOS NUCIFERA) and 0.1% of lactobacillus/rye flour ferment filtrate. The granule composition used in this application example was the granule composition produced in Example 8.

The preparation method of the shampoo granules was the same as that of Example 8, and comprised the following steps:
S1. the components except the thickener were weighed according to the formulation, and then mixed evenly, dried, and ground into powder;
S2. the thickener was weighed according to the formulation, then mixed with water to give a thickener aqueous solution with a mass concentration of 0.5%;
S3. the powder obtained in step S1 was added to a granulator, and then the thickener aqueous solution obtained in step S2 was sprayed into the granulator, and a temperature of the air blown by a blower into the granulator was controlled at 85° C. with a speed of the blower of 15 Hz, so that the powder was turned up and down in the granulator to fully contact with the sprayed aqueous solution, thereby forming granules; wherein the outlet air temperature of the granulator was controlled at 30° C. and the humidity in the granulator was controlled at 30%; preferably, the thickener aqueous solution was evenly sprayed into the granulator by using a peristaltic pump;
S4. target granules within a 10-100 mesh range were sieved as the shampoo granules for filling to give the finished product.

### Comparative Application Examples 1-11

Solid shampooing granules prepared by the granule compositions obtained in Comparative Examples 1-11 were provided in these comparative application examples, which differed from the Application Example 1 only in that the granule composition obtained in Example 1 was replaced with the granule compositions obtained in Comparative Examples 1-11 respectively.

### Effect Example

### 1. Water Activity (Aw) Test:

The water activity of the granule compositions obtained in Examples 1-8, Comparative Examples 1-5 and 10 were tested. The test method was carried out using a water activity meter, according to United States Pharmacopeia-National Formulary (USP-NF) General Chapter 922: Water Activity. The results were shown in Table 3.

TABLE 3

| Sample | Water activity |
|---|---|
| Ex. 1 | 0.42 |
| Ex. 2 | 0.51 |
| Ex. 3 | 0.49 |
| Ex. 4 | 0.45 |
| Ex. 5 | 0.44 |
| Ex. 6 | 0.54 |
| Ex. 7 | 0.45 |
| Ex. 8 | 0.36 |
| CEx. 1 | 0.84 |
| CEx. 2 | 0.73 |
| CEx. 3 | 0.66 |
| CEx. 4 | 0.88 |
| CEx. 5 | 0.80 |
| CEx. 10 | 0.67 |

Note:
Ex. represents “Example”; and CEx. represents “Comparative Example”.

Water activity (Aw) refers to the availability or activity of water in a substance, which measures the degree to which water can participate in biochemical reactions in a system. In the preparation process of a solid cleansing and caring product including the granule composition, water activity has an important impact on the properties, stability and use effect of the product. In general, a cleansing and caring product with a lower water activity is more conducive to improving the stability of the product, avoiding the degradation or inefficiency of the active ingredients, and effectively inhibiting the growth and reproduction of microorganisms, thereby reducing the deterioration risk of the product. In addition, reduced water activity helps to control the generation of foam and achieve a more stable amount and quality of foam, so that the solid cleansing and caring product including the granule composition can generate more uniform foam which is easy to rinse without residue.

As can be seen from the results in Table 3 that the selection of surfactants has an important effect on the water activity of the granule compositions. The water activity of the solid granule compositions obtained in this disclosure reaches 0.36-0.54, which is significantly lower than those in the comparative examples, so that a balance of the granule compositions in aspects such as cleansing ability, foam stability and use effect is realized, and the stability and performance of the granule compositions are effectively ensured. However, in Comparative Examples 1-5, one or two of the surfactants, such as distearyl dimethyl ammonium chloride, sodium C14-16 olefin sulfonate, and sodium cocoyl hydroxyethyl sulfonate and sodium lauroyl glutamate, are absent, and in Comparative Example 10, distearyl dimethyl benzyl quaternary ammonium chloride is replaced with octadecyl dimethyl benzyl quaternary ammonium chloride, the water activity of the granule compositions obtained in Comparative Examples 1-5 and 10 is significantly increased in a case where the same preparation process is adopted, which leads to the problems such as deliquescence or deterioration of the granule compositions, and the user experience is worse than that of the examples.

### 2. Stability Test:

The solid granule compositions obtained in Examples 1-8 were tested for their stability, and the test method was as follows.

The internal control standard used in this disclosure was higher than the Chinese national standards and light industry standards. According to the internal control standard, the sample was stored at a high temperature of 45±1° C. for 2 months, and at a low temperature of −18±1° C. for an additional 2 months; then the sample was subjected to a three-cycle high-low temperature test, each cycle consisting of 24-hour storage at a high temperature of 45±1° C. followed by 24-hour storage at a low temperature of −18±1° C. The results were shown in Table 4.

TABLE 4

| | | | | |
|---|---|---|---|---|
| | | | | Three-cycle high-low |
| Sample | Test conditions | 45 ± 1° C. | −18 ± 1° C. | temperature test |

| | | | |
|---|---|---|---|
| Ex. | Initial | Appearance | White or yellow granules |
| 1-8 | | pH (an aqueous solution | 6-8 |

of the sample with a
weight ratio of the sample
to water of 1:10) at room
temperature

| | |
|---|---|
| After test | The granules showed no significant change. |
| Conclusion | Qualified |

Note:
Ex. represents “Example”.

As can be seen from the results in Table 4 that the solid granule compositions obtained in this disclosure have a significant stability advantage compared with the traditional liquid cleansing and caring products, indicating that solid granules, instead of traditional liquid cleansing and caring products, can significantly improve the stability of the product. The form of solid granules can not only maintain the efficacy of cleansing and caring products, but also has obvious advantages in environmental protection, economy, convenience and energy saving.

### 3. Soothing Effect Test:

The solid granule compositions obtained in Examples 1-8, Comparative Examples 6-9 and 11 were tested for their soothing effect, and the test method was as follows.

Experimental principle: relevant literature shows that hyaluronic acid is widely present in skins and other tissues and has a very close relationship with the skin. The macromolecule hyaluronic acid can inhibit inflammatory allergic reactions, thus relieving the discomfort caused by skin allergies. The macromolecule hyaluronic acid will be degraded by the hyaluronidase to generate a degradation product N-acetylglucosamine, resulting in a decrease in the amount of the macromolecule hyaluronic acid. Therefore, the activity of hyaluronidase can be indirectly reflected by the level of N-acetylglucosamine in the reaction system. The anti-sensitivity activity of a sample was evaluated through in vitro hyaluronidase activity inhibition experiments. Inhibition of hyaluronidase activity can reduce the degradation of hyaluronic acid and reduce the sensitivity and discomfort of the skin.

Test method: the test sample was mixed with a hyaluronidase solution to give a mixed solution with a concentration of the sample of 5 w %; the mixed solution was incubated at 37° C. for 10 min after shaking and mixing, a sodium hyaluronate solution was added so that the test sample was incubated at 37° C. for 45 min; the BSA (Bovine Serum Albumin) solution was added so that the test sample was further incubated for 10 min, and the OD (Optical Density) value at 600 nm was detected. The hyaluronidase inhibition rate was calculated according to the following equation. The results were shown in Table 5.

$$Hyaluronidase inhibition rate ( \% ) = ( 1 - OD value of sample / OD value of control ) \times 100 \%$$

wherein, the OD value of sample was detected after the test sample was added, and the OD value of control was detected before the test sample was added.

TABLE 5

| | Hyaluronidase |
|---|---|
| Sample | inhibition rate % |
| Ex. 1 | 84 |
| Ex. 2 | 72 |
| Ex. 3 | 77 |
| Ex. 4 | 78 |
| Ex. 5 | 81 |
| Ex. 6 | 82 |
| Ex. 7 | 70 |
| Ex. 8 | 87 |
| CEx. 6 | 58 |
| CEx. 7 | 56 |
| CEx. 8 | 61 |
| CEx. 9 | 42 |
| CEx. 11 | 64 |

Note:
Ex. represents “Example”; CEx. represents “Comparative Example”.

As can be seen from the results shown in Table 5 that the granule compositions obtained in this disclosure have a good soothing effect, can effectively relieve and eliminate the discomfort of skin or scalp such as irritation or dryness caused by deep cleansing ingredients, provide sedation, enhance the skin protection ability, and reduce the barrier damage caused by the deep cleansing ingredients, so that the product is milder and more comfortable during use. However, in Comparative Examples 1-6, in which any one or two of dipotassium glycyrrhizinate, the chamomile extract and the calendula extract are absent, the hyaluronidase inhibition rate of the prepared granule compositions is significantly reduced compared with that in the examples. In Comparative Example 11, in which the chamomile extract is replaced with the aloe vera extract, the hyaluronidase inhibition rate of the granule composition is also decreased. That is, the soothing effect of the compositions in the comparative examples is significantly worse, indicating that a better synergistic effect can be achieved only if the dipotassium glycyrrhizinate, the chamomile extract and the calendula extract, which are selected as the soothing ingredient in this disclosure, cooperate with one another, thereby synergically enhancing the soothing effect of the compositions of the present disclosure and effectively avoiding the discomforts caused by deep cleansing.

### 4. Foaming Performance Test:

Solid shampoo granules obtained in Application Examples 1-8, and Comparative Application Examples 1-5 and 10 were tested for their foaming performance and foam stability, and the method was as follows.

The foam performance of the sample was evaluated by using a Rothschild foam meter: a sample solution with a concentration of 0.2 wt % was prepared with 2.5 g of the shampoo granules obtained in each of Application Examples 1-8, and Comparative Application Examples 1-5 and 10. 200 mL of the sample solution was flowed through a narrow hole with an inner diameter of 2.9 mm at a height of 900 mm down into 50 mL of the sample solution with the same temperature and concentration. After 200 mL of the sample solution ran out, the foam height was recorded as an evaluation index of the foaming ability of the sample. After 5 min, the foam height was recorded as an evaluation index of the foam stability. The test was repeated three times, and the average value for each sample was calculated. The results were shown in Table 6.

TABLE 6

| | Foam | Foam height |
|---|---|---|
| Sample | height/cm | after 5 min/cm |
| AEx. 1 | 13.9 | 13.4 |
| AEx. 2 | 9.3 | 8.5 |
| AEx. 3 | 11.4 | 10.8 |
| AEx. 4 | 12.3 | 11.6 |
| AEx. 5 | 12.8 | 12.2 |
| AEx. 6 | 9.0 | 8.3 |
| AEx. 7 | 13.5 | 13.0 |
| AEx. 8 | 14.3 | 13.6 |
| CAEx. 1 | 7.7 | 6.5 |
| CAEx. 2 | 5.8 | 4.3 |
| CAEx. 3 | 6.5 | 5.5 |
| CAEx. 4 | 4.1 | 2.8 |
| CAEx. 5 | 4.9 | 4.0 |
| CAEx. 10 | 7.9 | 6.8 |

Note:
AEx. represents “Application Example”; and CAEx. represents “Comparative Application Example”.

In general, the foaming performance and foam stability have a certain impact on the cleansing efficacy of a cleansing and caring product. Rich and stable foam can effectively enhance the application process, thereby improving the removal of dirt and oils from the surface of skin or hair. Besides, it also contributes to the continuity of the cleansing process, especially in the long massage process, to ensure the product's cleansing effect on scalp or hair, thus significantly enhancing the cleansing and oil control efficacy and use experience of the product.

As can be seen from the results shown in Table 6 that the shampoo granules obtained in Application Examples 1-8 of the disclosure have richer foam and better foam stability. However, in Comparative Application Examples 1-5 in which any one or two surfactants in the surfactant blend defined in the disclosure are absent, and in Comparative Application Example 10 in which the distearyl dimethyl ammonium chloride is replaced, the foam amount and foam stability of the prepared shampoo granules are significantly reduced, that is, the cleansing performance and usage experience of the shampoo granules are significantly worse than those of the examples.

The sole FIGURE shows the foaming effect of the shampoo granule obtained in Application Example 1. As can be seen from the sole FIGURE that when the shampoo granule is rubbed with a little of water, the foam becomes richer and richer, and the foam is fine and stable.

### 5. Rinsing Performance Test:

The solid shampoo granule obtained in Application Example 1, as a test sample, was tested for its rinsing performance, and a commercially available shampoo was taken as a control sample. The method was as follows.

The subjects' hands were rinsed with water with a pH value of 7 until the pH values of the water on the palms of both hands were about 7. Then 2 g of the test sample and 2 g of the control sample were respectively placed on the palms and each sample was rubbed with an equal volume of water for 30 seconds, the hands were rinsed with water with a pH value of 7 under the same water flow rate for 10 seconds, and then the pH values of residual water on the palms were measured by a pH meter. In this way, the rinsing performance of the sample was evaluated. The test was repeated 5 times and then an average value was calculated. The results were shown in Table 7.

TABLE 7

| Sample | | pH value |
|---|---|---|
| Shampoo | pH value of the water | 6.2 ± 0.14 |
| granule of | on the palm after | |
| AEx. 1 | rubbing sample for 30 | |
| | seconds | |
| | pH value of the water | 7.0 ± 0.08 |
| | on the palm after | |
| | rinsing for 10 seconds | |
| Commercially | pH value of the water | 5.5 ± 0.17 |
| available | on the palm after | |
| shampoo | rubbing sample for 30 | |
| | seconds | |
| | pH value of the water | 5.8 ± 0.12 |
| | on the palm after rinsing | |
| | for 10 seconds | |

Note:
AEx. represents “Application Example”.

As can be seen from the results shown in Table 7 that compared with the traditional shampoo, the shampoo granule prepared by the granule composition of this disclosure can be quickly dissolved in water, achieving excellent rinsing performance without residue and significantly reducing the water consumption, while the traditional shampoo requires more water to completely remove the residue when rinsing. Therefore, the shampoo granule of this disclosure has advantages over traditional shampoo in terms of water consumption, environmental protection and portability.

### 6. Product's Sensory Performance Test:

The shampoo granules obtained in Application Examples 1-8 and Comparative Application Examples 1-11 were tested for their sensory performance.

190 subjects with no skin abnormalities between the ages of 20 and 60 were randomly selected, including 82 males and 108 females. The subjects were randomly divided into 19 groups with 10 people in each group. The samples in Application Examples 1-8 and Comparative Application Examples 1-11 were trialed respectively, and evaluated for usage experience. The samples were each trialed for 2 weeks. Based on their usage experience, the subjects evaluated the samples based on a scoring system. Evaluation items included cleansing and oil control efficacy, foam richness, easy rinsing without residue, soothing and moisturizing effects. A 10-point scale was adopted for scoring, 1 score means the worst and 10 scores mean the best. The evaluation results were represented by the average scores of all subjects in each group, as shown in Table 8. The higher the average score, the better the performance.

TABLE 8

| | Cleansing | | Easy | Soothing | | Cleansing | | easy | soothing |
|---|---|---|---|---|---|---|---|---|---|
| | and oil | | rinsing | and | | and oil | | rinsing | and |
| | control | Foam | without | moisturizing | | control | Foam | without | moisturizing |
| Sample | efficacy | richness | residue | effects | Sample | efficacy | richness | residue | effects |
| AEx. 1 | 9.6 | 9.8 | 9.8 | 9.5 | CAEx. 3 | 7.9 | 8.0 | 8.2 | 7.5 |
| AEx. 2 | 8.8 | 9.3 | 9.4 | 8.9 | CAEx. 4 | 5.2 | 5.4 | 6.3 | 8.3 |
| AEx. 3 | 9.3 | 9.4 | 9.5 | 9.0 | CAEx. 5 | 6.3 | 6.2 | 7.2 | 7.9 |
| AEx. 4 | 9.1 | 9.3 | 9.6 | 9.2 | CAEx. 6 | 8.3 | 8.2 | 8.7 | 7.1 |
| AEx. 5 | 9.4 | 9.5 | 9.6 | 9.3 | CAEx. 7 | 8.4 | 8.3 | 8.5 | 6.9 |
| AEx. 6 | 8.6 | 9.2 | 9.3 | 9.4 | CAEx. 8 | 8.1 | 8.2 | 8.6 | 7.3 |
| AEx. 7 | 9.5 | 9.6 | 9.6 | 8.6 | CAEx. 9 | 8.2 | 8.3 | 8.5 | 5.9 |
| AEx. 8 | 9.7 | 9.9 | 9.8 | 9.7 | CAEx. 10 | 7.8 | 8.0 | 7.9 | 7.8 |
| CAEx. 1 | 7.6 | 7.9 | 8.1 | 8.1 | CAEx. 11 | 8.4 | 8.3 | 8.5 | 7.5 |
| CAEx. 2 | 6.7 | 6.4 | 7.4 | 8.2 | | | | | |
| CAEx. 3 | 7.9 | 8.0 | 8.2 | 7.5 | | | | | |

Note:
AEx. represents “Application Example”; and CAEx. represents “Comparative Application Example”.

The results in Table 8 show that the shampoo granules prepared by the granule composition of the disclosure can achieve an effective balance in aspects of cleansing and oil control efficacy, foaming performance, rinsing performance, moisturizing and soothing effect, and significantly reduce water consumption during use. In particular, the shampoo granules obtained in Application Examples 1 and 8 have the best comprehensive performance and can provide users with a more ideal experience. However, in Application Comparative Examples 1-5 and 10, because of the absence of components in the specified surfactant blend, the cleansing and oil control efficacy and foam quality of the shampoo granules are significantly affected; in Comparative Application Examples 6-9 and 11, because of the absence of dipotassium glycyrrhizinate, chamomile extract or calendula extract, the soothing and moisturizing effect of shampoo granules are significantly reduced, and thus a certain degree of dryness, tightness, or mild irritation occurs after the scalp is cleaned.

Finally, it should be noted that the above embodiments are intended to illustrate the technical solutions of the disclosure and not to limit the protection scope thereof. Although the disclosure is described in detail by reference to preferred embodiments, persons of ordinary skill in the art should understand that the technical solution of the disclosure can be modified or equivalently replaced without deviating from the substance and scope of the technical solution of the disclosure.

What is claimed is:

1. A granule composition, comprising the following components in parts by weight: 21-73 parts of a surfactant blend, 2.1-21 parts of a humectant, 1-4.9 parts of a soothing ingredient, 10-20 parts of a filler, 1-5 parts of an anti-caking agent and 0.1-1 part of a thickener; wherein, the surfactant blend comprises a cationic surfactant, an anionic surfactant and an amino acid surfactant;
the cationic surfactant comprises at least one of distearyl dimethyl ammonium chloride and guar gum hydroxypropyl trimethyl ammonium chloride; the anionic surfactant comprises sodium cocoyl hydroxyethionate and sodium C14-16 olefin sulfonate; and the amino acid surfactant comprises sodium lauryl glutamate.

2. The granule composition according to claim 1, wherein the granule composition comprises the following components in parts by weight: 30.5-57.5 parts of the surfactant blend, 8.3-18.8 parts of the humectant, 1.9-3.7 parts of the soothing ingredient, 15-20 parts of the filler, 2-4 parts of the anti-caking agent and 0.2-0.8 part of the thickener.

3. The granule composition according to claim 2, wherein the granule composition comprises the following components in parts by weight: 50.5 parts of the surfactant blend, 14.5 parts of the humectant, 2.6 parts of the soothing ingredient, 20 parts of the filler, 3 parts of the anti-caking agent and 0.5 part of the thickener.

4. The granule composition according to claim 1, wherein the surfactant blend is a formulated blend of distearyl dimethyl ammonium chloride, sodium C14-16 olefins sulfonate, sodium cocoyl hydroxyethylsulfonate and sodium lauroyl glutamate; and
a mass ratio of the distearyl dimethyl ammonium chloride/sodium C14-16 olefin sulfonate/sodium cocoyl hydroxyethionate/sodium lauroyl glutamate is (0.1-3):(1-10):(15-35):(5-25).

5. The granule composition according to claim 4, wherein the mass ratio of the distearyl dimethyl ammonium chloride/sodium C14-16 olefin sulfonate/sodium cocoyl hydroxyethionate/sodium lauroyl glutamate is 2:3.5:35:10.

6. The granule composition according to claim 1, wherein the soothing ingredient comprises allantoin, dipotassium glycyrrhizinate, a chamomile extract and a calendula extract; and
a mass ratio of allantoin/dipotassium glycyrrhizinate/the chamomile extract/the calendula extract is (0.5-2):(0.3-0.9):(0.1-1):(0.1-1).

7. The granule composition according to claim 1, wherein the humectant comprises sodium hyaluronate, trehalose and sorbitol;
and/or, the filler comprises a modified corn starch;
and/or, the anti-caking agent comprises magnesium stearate;
and/or, the thickener comprises hydroxypropyl methyl cellulose.

8. The granule composition according to claim 1, wherein the granule composition further comprises the following components in parts by weight: 0.2-0.8 part of a pH regulator and 0.5-1.5 parts of a fragrance.

9. The granule composition according to claim 8, wherein the pH regulator comprises citric acid; and/or, the fragrance comprises an essence.

10. A cleansing and caring product, comprising the granule composition according to claim 1.

11. The cleansing and caring product according to claim 10, wherein the cleansing and caring product is selected from a shampoo product, a facial cleansing product and a bath product.

12. A shampoo granule with deep cleansing, soothing and smoothing properties, wherein the shampoo granule comprises the granule composition according to claim 1, a softener and a hair regulator.

13. The shampoo granule according to claim 12, wherein the softener comprises at least one of acanthus kernel oil, Jojoba seed oil and coconut oil; and
the hair regulator comprises lactobacillus/rye flour ferment filtrate.

14. The granule composition according to claim 4, wherein the granule composition further comprises the following components in parts by weight: 0.2-0.8 part of a pH regulator and 0.5-1.5 parts of a fragrance.

15. The granule composition according to claim 6, wherein the granule composition further comprises the following components in parts by weight: 0.2-0.8 part of a pH regulator and 0.5-1.5 parts of a fragrance.

16. The granule composition according to claim 7, wherein the granule composition further comprises the following components in parts by weight: 0.2-0.8 part of a pH regulator and 0.5-1.5 parts of a fragrance.

17. A cleansing and caring product, comprising the granule composition according to claim 4.

18. A cleansing and caring product, comprising the granule composition according to claim 6.

19. A cleansing and caring product, comprising the granule composition according to claim 7.

20. A cleansing and caring product, comprising the granule composition according to claim 8.
